(19) United States
(12) Patent Application Publication
(10) Pub. No.: US 2003/0169888 A1
(43) Pub. Date: Sep. 11, 2003

(54) Frequency dependent acoustic beam forming and nulling

(76) Inventors: Nikolas Subotic, Ann Arbor, MI (US); Christopher Roussi, Kalamazoo, MI (US); Joseph Burns, Ann Arbor, MI (US)

Correspondence Address: John G. Posa, Gifford, Krass, Groh, Sprinkle,, Anderson & Citkowski, P.C., 280 N. Old Woodward Ave., Suite 400, Birmingham, MI, 48009-5394, US

(21) Appl. No.: 10/385,067

(22) Filed: Mar. 10, 2003

Related U.S. Application Data
(60) Provisional application No. 60/362,688, filed on Mar. 8, 2002.

(51) Int. Cl. A61F 11/06; G10K 11/16; H03B 29/00

(52) U.S. Cl. 381/71.4

(57) ABSTRACT

Broadly, this invention resides in apparatus and methods involving a set of soundfield nulling algorithms providing a localized decrease in sound intensity. Among the benefits of the approach, is that there is little, if any, affect on other important positions such as power or spectral content, insofar as energy is directed to unimportant areas. In the preferred embodiment, two separate algorithms are used, depending upon the frequency range of the acoustic signal. For lower frequencies (for example, less than 300 Hz), the algorithm is based on Cepstral techniques and overtly uses the fact that in an enclosed area, the predominant acoustic influence is in the form of standing waves. At higher frequencies, however, (i.e., 300 Hz and above), the sound is due to free-space propagation. Consequently, single free-space algorithms that are applied across the spectrum have great difficulty in providing useful sound nulls without distortion.

### REFERENCE TO RELATED APPLICATION

This application claims priority from U.S. Provisional Patent Application Serial No. 60/362,688, filed Mar. 8, 2002, the entire content of which is incorporated herein by reference.

### FIELD OF THE INVENTION

This invention relates generally to sound and noise cancellation and, in particular, to space/time processing for sound field nulling.

### BACKGROUND OF THE INVENTION

With all of the different forms of entertainment now available in automobiles and other vehicles, cross-talk and other forms of interference have become increasingly problematic. Even within the same vehicle, a problem represents when one person does not wish to hear the entertainment being enjoyed by another passenger. Not only do the signals compete with one another, but distraction to the driver may also occur.

As discussed in U.S. Pat. No. 4,977,600, prior-art sound attenuators include passive as well as active attenuators. The use of sound absorbing material is a well-known passive attenuating technique. Active sound attenuators have taken two general approaches. The first is to attenuate the sound at its source. This generally includes measuring the sound at its source and producing a canceling sound 180-degree out-of-phase at the source of the sound or noise. The second method is to cancel or attenuate the noise at a location, remote from the source of the noise, at which inhabitants are expected to occupy.

Within the second group of active sound attenuators in which the noise is cancelled or attenuated at a remote point from the source of the noise, two general overall methodologies have been developed. In the first methodology, noise is attenuated throughout the total enclosure. This generally would include measuring the noise level within the enclosure and providing appropriate canceling noise to cancel the noise throughout the total enclosure. The less sophisticated systems use a few actuators to produce the canceling noise where others do a complete study of the total enclosure finding the nodal points of maximum noise and placing the actuators at the maximum nodal point.

This second system requires a substantial amount of time and research to determine the nodal points. This method and the less sophisticated systems depend on noise produced during a test period. The noise itself may have different nodal points or be noise different from that designed around and therefore, the anti-noise or canceling signal produced by the actuators may not be effective. Also, the canceling noise may combine with the noise level instead of canceling and reducing it.

In addition to the dynamics of the enclosure, the interaction of the actuators must also be taken into account. This is especially true where the actuators are substantially displaced from the sensors and the actuator must be driven at sufficiently high amplitude. This substantially increases the complexity of the noise patterns within the enclosure.

A second methodology of canceling the noise in an enclosure specifically at the location of the occupant or inhabitant includes placing earphones on the occupant. The earphones not only operate as a passive device for canceling sound, they may also have actuators and sensors which measure and actively cancel the noise at the ears. These have generally been suggested for use in industrial environments where there are high levels of noise due to machinery or where a headset is naturally worn, for example by pilots.

In vehicles, which comprise an enclosure, or other space, it is highly desirable to cancel noise existing near the occupants produced by known sources of noise, for example, an engine or other periodically occurring noises of the vehicle, without adversely affecting the hearing of the driver or other occupant(s). Indeed, it is illegal in some states to wear earphones or other devices while driving since it is believed that it impairs the driver and other occupants from hearing emergency vehicles or being aware of other dangerous conditions about them. Thus, cancellation of the noise in the total enclosure has been the general approach to noise attenuation within the interior of the vehicle.

Fortunately, however, the specific features of entertainment nulling separate the problem from the more general class of active noise cancellation. For one thing, the sound generated by the various entertainment systems within a vehicle represent known signals such that the propagation and standing-wave environment associated therewith may be measurable, modelable, or both. The emitter locations are also physically known, enabling space/time filters to be tuned to position, frequency response, multi-path and/or signal source.

### SUMMARY OF THE INVENTION

Broadly, this invention resides in apparatus and methods involving a set of sound field nulling algorithms providing a localized decrease in sound intensity. Among the benefits of the approach, is that there is little, if any, affect on other important positions such as power or spectral content, insofar as energy is directed to unimportant areas. In the preferred embodiment, two separate algorithms are used, depending upon the frequency range of the acoustic signal. For lower frequencies (for example, less than 300 Hz), the algorithm is based on Cepstral techniques and overtly uses the fact that in an enclosed area, the predominant acoustic influence is in the form of standing waves. At higher frequencies, however, (i.e., 300 Hz and above), the sound is due to free-space propagation. Consequently, single free-space algorithms that are applied across the spectrum have great difficulty in providing useful sound nulls without distortion.

### BRIEF DESCRIPTION OF THE DRAWINGS

FIG. 1 is a diagram which helps to understand the unique aspects of the problem solved by this invention;

FIG. 2 is a drawing which has a particular emphasis on the processing approach for high frequencies;

FIG. 3 is a diagram which shows how linear filters may be used on each source to provide full connectivity between sources and speaker/output devices; and

FIG. 4 is a table which lists unique aspects of the approach.

### DETAILED DESCRIPTION OF THE INVENTION

Broadly, this invention resides in apparatus and methods involving a set of soundfield nulling algorithms providing a localized decrease in sound intensity. Among the benefits of the approach, is that there is little, if any, affect on other important positions such as power or spectral content, insofar as energy is directed to unimportant areas.

In the preferred embodiment, two separate algorithms are used, depending upon the frequency range of the acoustic signal. For lower frequencies (for example, less than 300 Hz), the algorithm is based on Cepstral techniques and overtly uses the fact that in an enclosed area, the predominant acoustic influence is in the form of standing waves.

At higher frequencies, however, (i.e., 300 Hz and above), the sound is due to free-space propagation. Consequently, single free-space algorithms that are applied across the spectrum have great difficulty in providing useful sound nulls without distortion.

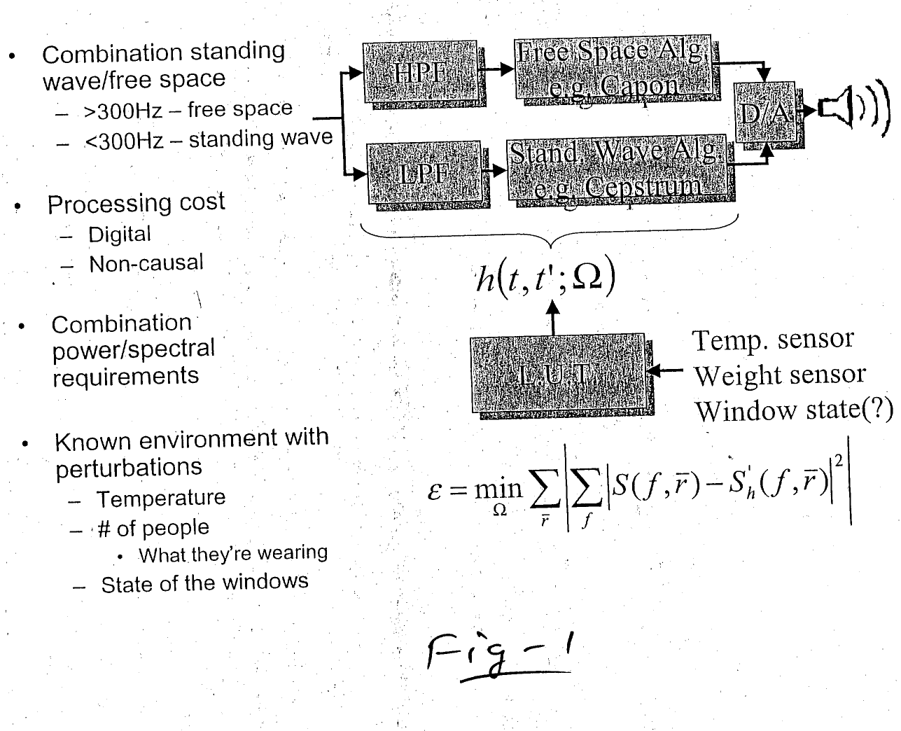

FIG. 1 is a diagram which helps to understand the unique aspects of the problem. Using high- and/or low-pass filtering, standing-wave and free-space algorithms are applied independently to a nulling environment, with the results being combined in a digital-to-analog conversion apparatus prior to acoustic transformation. Although a boundary of 300 Hz is being used herein as a transition point between the standing-wave and free-space algorithmic separation, it will be appreciated that this particular frequency is not fixed, but that another frequency or frequencies may be used as transition points.

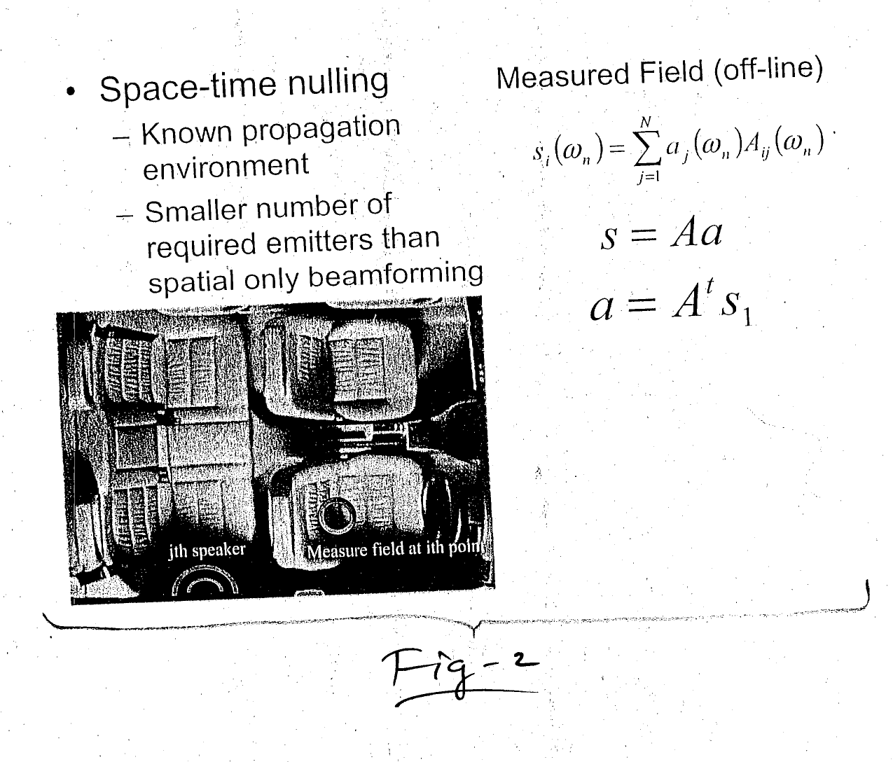

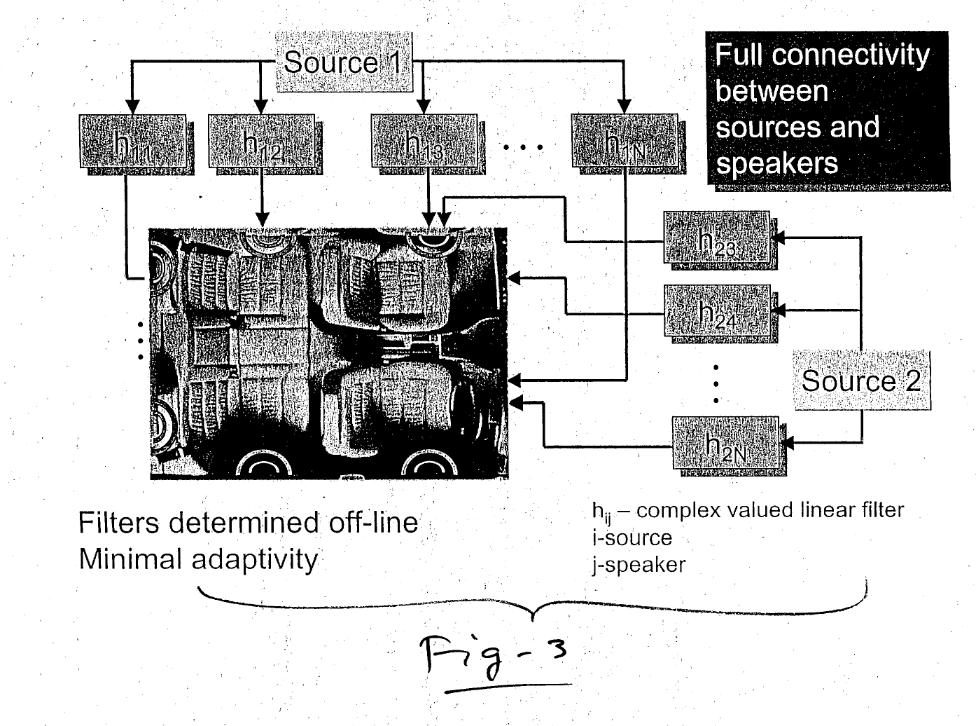

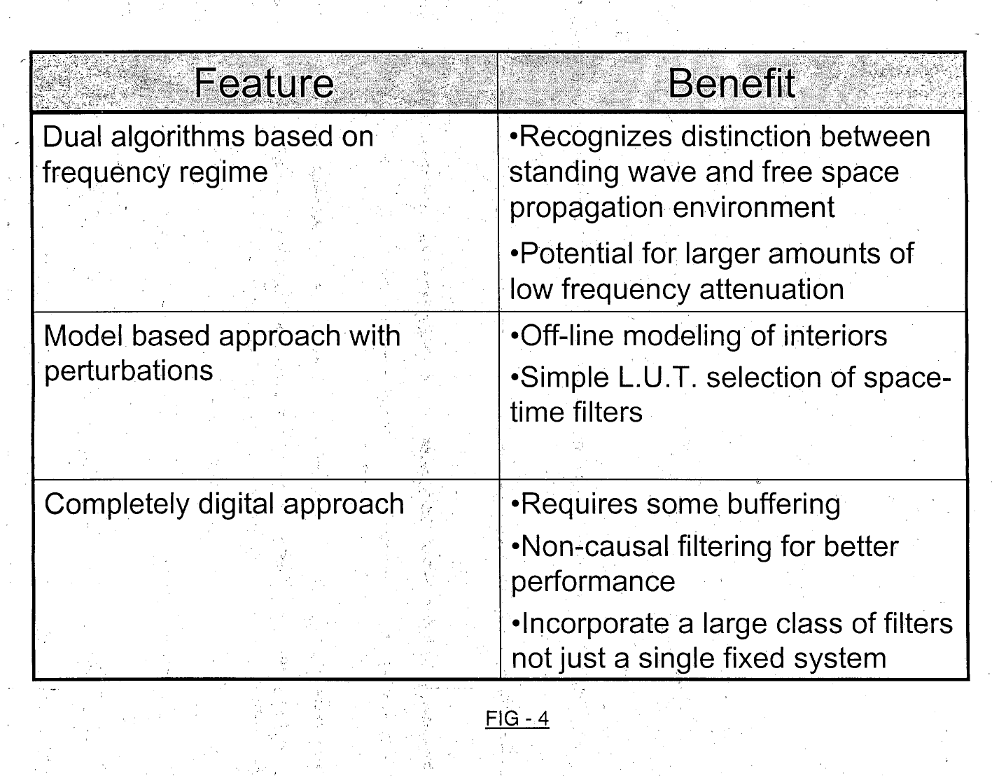

To assist in an accurate cancellation, variables are preferably provided in association with temperature, the number of people, the state/position of windows and other features to enhance accuracy. FIG. 2 is a drawing which has a particular emphasis on the processing approach for high frequencies. FIG. 3 is a diagram which shows how linear filters may be used on each source to provide full connectivity between sources and speaker/output devices. FIG. 4 is a table which lists unique aspects of the approach.

We claim:

1. A method of soundfield nulling, comprising the steps of:
designating a transition frequency or region below which there are lower frequencies to be nulled, and above which there are higher frequencies to be nulled;
canceling the lower frequencies using a first algorithm that considers standing waves; and
canceling the lower frequencies using a second algorithm that considers free-space propagation.

2. The method of claim 1, wherein the first algorithm includes a Cepstral technique.

3. The method of claim 1, wherein the second algorithm includes a Capon technique.

4. The method of claim 1, including a transition frequency of around 300 Hz.

5. The method of claim 1, wherein one or more of the following are taken into account to improve the cancellation effect:
ambient temperature;
characteristics of the listener or nearby individuals; and
enclosure physical features.

6. The method of claim 1, wherein the algorithms are applied to an enclosed space.

7. The method of claim 6, wherein the enclosed space comprises a vehicle interior.

8. The method of claim 1, further including the steps of:
receiving an audible signal to be nulled;
low-pass and/or high-pass filtering the signal to separate out the lower and higher frequencies;
applying the algorithms to their respective frequency ranges; and
generating an acoustical signal based upon the result.

9. Sound field nulling apparatus, comprising:
an input for receiving an audible signal to be nulled;
frequency-based filtering to separate out lower and higher frequencies from the audible signal;
a processor operative to apply first and second sound-cancellation algorithms to the lower and higher frequencies; and
an output for generating an acoustical signal based upon the result.

10. The apparatus of claim 9, wherein the first algorithm includes a Cepstral technique.

11. The apparatus of claim 9, wherein the second algorithm includes a Capon technique.

12. The apparatus of claim 9, wherein the lower and higher frequencies are below and above about 300 Hz.

13. The apparatus of claim 9, further including one or more sensors to detect one or more of the following to assist the processor in applying one or both of the sound-cancellation algorithms:
ambient temperature;
characteristics of the listener or nearby individuals; and
enclosure physical features.
